(19) United States
(12) Patent Application Publication
(10) Pub. No.: US 2013/0078613 A1
(43) Pub. Date: Mar. 28, 2013

(54) Selection of HCV Treatment

(71) Applicants: Victor Iglesias, Merzhausen (DE); David Ipe, San Carlos, CA (US); James Thommes, San Francisco, CA (US); Yonghong Zhu, Mountain View, CA (US)

(72) Inventors: Victor Iglesias, Merzhausen (DE); David Ipe, San Carlos, CA (US); James Thommes, San Francisco, CA (US); Yonghong Zhu, Mountain View, CA (US)

(73) Assignee: ROCHE MOLECULAR SYSTEMS, Pleasanton, CA (US)

(21) Appl. No.: 13/433,571

(22) Filed: Mar. 29, 2012

Related U.S. Application Data
(60) Provisional application No. 61/470,281, filed on Mar. 31, 2011.

(51) Int. Cl. C12Q 1/68

(52) U.S. Cl. CPC C12Q 1/6827
USPC 435/5

(57) ABSTRACT

The present invention is based on the discovery of an association between the SNP genotype at the rs12979860 locus in the IL28B gene promoter and the probability of achieving a SVR in HC V-infected patients that are given a triple therapy treatment that contains peginterferon alfa-2a, ribavirin, and a HCV NS5B polymerase inhibitor.

### CROSS REFERENCE TO RELATED INVENTION

This application claims the benefit of priority of U.S. Provisional Patent Application Ser. No. 61/470,281, filed Mar. 31, 2011, which is incorporated herein by reference in its entirety.

### FIELD OF THE INVENTION

The present invention relates to methods that are useful for selecting appropriate pharmacological treatment regimens for patients infected with Hepatitis C Virus (HCV).

### BACKGROUND OF THE INVENTION

Hepatitis C virus (HCV) is responsible for a large proportion of chronic liver disease worldwide and accounts for 70% of cases of chronic hepatitis in industrialized countries. The global prevalence of chronic hepatitis C (CHC) is estimated to average 3% (ranging from 0.1%-5%), with an estimated 170 million chronic carriers worldwide (2.7 million in the USA and 5 million in Western Europe [1, 2]. Approximately one-fifth of chronically infected patients with HCV eventually develop cirrhosis, which may lead to liver failure and hepatocellular carcinoma [2]. HCV-related liver failure is one of the primary reasons for liver transplantation today.

The current standard of care (SOC) for treatment-naïve patients with chronic hepatitis C infection is combination therapy with polyethylene glycol conjugated interferon alpha (PEG-IFN) plus ribavirin (RBV) [3]. In patients chronically infected with HCV genotype 1, which represents the majority of HCV-infected patients, responses to PEG-IFN remain sub-optimal with sustained virologic response (SVR) rates between 42-52% [4, 5. 6].

There is a substantial need for new therapeutic options for this patient population.

Interferon alpha (IFN) was the first drug shown to have bioactivity against HCV. Hoffmann-La Roche Inc. has chemically modified the interferon alfa-2a molecule by covalently attaching a branched methoxy polyethylene glycol moiety [9]. PEG-IFN has a decreased systemic clearance rate and an approximately 10-fold increase in serum half-life compared with interferon alfa-2a, and as a result PEG-IFN circulates in the blood much longer than the parent compound. Subsequent evaluation of PEG-IFN, 180 μg once weekly (qw), in three large clinical trials in over 1400 patients showed that treatment with PEG-IFN was more efficacious than treatment with IFN thrice weekly [10].

Ribavirin is a guanosine analogue that inhibits the in vitro replication of a wide range of RNA and DNA viruses [11]. The mechanism by which RBV acts as an antiviral is not fully defined, although it may involve alteration of cellular nucleotide pools and inhibition of viral RNA synthesis [12]. RBV monotherapy has little or no effect on the replication of HCV, but it can result in normalization of serum alanine aminotransferase (ALT) activity and improvement in liver histology. However, relapse occurs in nearly all patients treated with RBV alone [13, 14]. Combining RBV with PEG-IFN has been found to be more effective than PEG-IFN monotherapy in the treatment of CHC. In a large clinical trial of 1121 patients of all genotypes, a sustained virologic response (SVR) was achieved in 53% of patients treated with PEG-IFN plus RBV as compared to 29% of patients treated with PEG-IFN alone [10].

The probability of achieving a Sustained Virological Response (SVR) varies with a collection of patient and viral factors. For example, younger patients, Caucasian and Asian patients, and individuals without advanced hepatic fibrosis are more likely to clear HCV infection after treatment [15-18] Similarly, patients infected with HCV genotypes 2 or 3, rather than genotype 1, and those with low baseline HCV RNA levels in serum have the best chance of a cure [4-6, 16, 18].

More precise prediction of SVR is currently possible only after the start of treatment. Regardless of HCV genotype, individuals who clear HCV RNA after 4 or 12 weeks of treatment have a much better chance of achieving an SVR than those with persistent viremia [19]. Rapid virological response (RVR, undetectable HCV RNA at week 4) is a strong predictor of SVR; conversely, failure to achieve an early virological response (EVR, greater than a two log decline in HCV RNA at week 12) is a strong predictor of nonresponse, independent of pretreatment characteristics [20].

The ability to prospectively differentiate between potential responders and non-responders to the standard of care could have a great impact on the care of patients with chronic hepatitis C. In addition to host and viral factors, host genetic diversity also influences the response to treatment with the standard of care [21]. Recent evidence from genome-wide association studies suggests that single nucleotide polymorphisms (SNPs) in the promoter region of the IL-28b gene, exert a strong influence on the probability of SVR in patients treated with peginterferon plus ribavirin [22-24].

RO5024048, also known as RG7128 or as mericitabine is a diisobutyl ester prodrug of RO4995855 and is a potent and selective nucleoside inhibitor of NS5B-directed HCV polymerase, with IC50 values of 0.61±0.04 μM [7]. Combination with PEG-IFN and RBV produces an additive antiviral effect. Activity has been demonstrated in vitro across the four main genotypes (1-4). Amino acid substitution in NS5B (S282T) has been identified through sequential passage of replicon cells to confer low level resistance to RO5024048, however no evidence of resistance was observed through phenotypic and sequencing analysis of clinical samples during mono- or combination therapy. Following oral administration, RO5024048 is rapidly absorbed and extensively converted to RO4995855, which is then phosphorylated to its active form. Both the nonclinical and early clinical assessment of RO5024048 supports its promise as an effective and selective antiviral drug for the treatment of HCV infection.

### SUMMARY OF THE INVENTION

The present invention is based on the discovery of an association between the SNP genotype at the rs12979860 locus in the IL28B gene promoter and the probability of achieving a SVR in patients that are given a triple therapy treatment that comprises peginterferon, ribavirin, and a HCV NS5B polymerase inhibitor.

In one embodiment, the invention provides for a method of selecting a duration of time of triple therapy treatment for achieving SVR in a human subject infected with HCV Genotype-1 or Genotype-4, comprising providing a sample from said human subject and identifying the nucleotide present at single nucleotide polymorphism rs12979860, wherein if said subject carries two C alleles at rs12979860, a duration of between 8 weeks and 12 weeks of said triple therapy is selected, and wherein if said subject does not carry two C alleles at rs12979860, a duration of at least 12 weeks and up to 48 weeks of said triple therapy treatment is selected.

In another embodiment, the invention provides for a method of selecting a duration of triple therapy treatment with or without subsequent standard of care (SOC) treatment for achieving SVR in a human subject infected with HCV Genotype-1 or Genotype-4, comprising providing a sample from said human subject and identifying the nucleotide present at single nucleotide polymorphism rs12979860, wherein if said subject carries two C alleles at rs12979860, a duration of between 8 weeks and 12 weeks of said triple therapy with or without subsequent 12 weeks of SOC treatment is selected, and wherein if said subject does not carry two C alleles at rs12979860, a duration of at least 12 weeks and up to 48 weeks of said triple therapy treatment with or without subsequent SOC treatment is selected.

### BRIEF DESCRIPTION OF THE DRAWINGS

FIG. 1 Study design of Clinical Study NV20536 (A) and NV22621 (B).

FIG. 2 Comparison of results from patients in NV20536 Group C with patients in NV22621 Group A.

### DETAILED DESCRIPTION OF THE INVENTION

### Definitions

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The term “response” to treatment is a desirable response to the administration of an agent. Virological endpoints included “early virological response” (EVR), defined as ≧2-log drop in serum HCV RNA from baseline to week 12 (by Cobas Amplicor HCV Monitor Test, v2.0, limit of quantitation 600 IU/mL), complete EVR (cEVR) defined as undetectable HCV RNA (<15 IU/mL) in serum (by Cobas Amplicor HCV Test v2.0, limit of detection 50 IU/mL) or and “sustained virological response” (SVR), defined as undetectable HCV RNA (<15 IU/mL) at the end of either a 12-week (SVR-12) or a 24-week (SVR-24) untreated follow-up period. The term “sustained rapid virological response” (sRVR) refers to undetectable HCV RNA (<15 IU/mL) during the period of 4 weeks and 22 weeks of treatment. The term “extended rapid virological response” (eRVR) refers to undetectable HCV RNA (<15 IU/mL) during the period of between 4 weeks and 20 weeks of treatment.

The terms “sample” or “biological sample” refers to a sample of tissue or fluid isolated from an individual, including, but not limited to, for example, tissue biopsy, plasma, serum, whole blood, spinal fluid, lymph fluid, the external sections of the skin, respiratory, intestinal and genitourinary tracts, tears, saliva, milk, blood cells, tumors, organs. Also included are samples of in vitro cell culture constituents (including, but not limited to, conditioned medium resulting from the growth of cells in culture medium, putatively virally infected cells, recombinant cells, and cell components).

The terms “interferon” and “interferon-alpha” (IFN) are used herein interchangeably and refer to the family of highly homologous species-specific proteins that inhibit viral replication and cellular proliferation and modulate immune response. Typical suitable interferons include, but are not limited to, recombinant interferon alpha-2b such as Intron® A interferon available from Schering Corporation, Kenilworth, N.J., recombinant interferon alpha-2a such as Roferon®-A interferon available from Hoffmann-La Roche, Nutley, N.J., recombinant interferon alpha-2C such as Berofor® alpha 2 interferon available from Boehringer Ingelheim Pharmaceutical, Inc., Ridgefield, Conn., interferon alpha-n1, a purified blend of natural alpha interferons such as Sumiferon® available from Sumitomo, Japan or as Wellferon® interferon alpha-n1 (INS) available from the Glaxo-Wellcome Ltd., London, Great Britain, or a consensus alpha interferon such as those described in U.S. Pat. Nos. 4,897,471 and 4,695,623 (especially Examples 7, 8 or 9 thereof) and the specific product available from Amgen, Inc., Newbury Park, Calif., or interferon alpha-n3 a mixture of natural alpha interferons made by Interferon Sciences and available from the Purdue Frederick Co., Norwalk, Conn., under the Alferon Tradename. The use of interferon alpha-2a or alpha-2b is preferred. Interferons can include pegylated interferons as defined below.

The terms “pegylated interferon”, “pegylated interferon alpha” and “peginterferon” (PEG-IFN) are used herein interchangeably and means polyethylene glycol modified conjugates of interferon alpha, preferably interferon alfa-2a and alfa-2b. Typical suitable pegylated interferon alpha include, but is not limited to, Pegasys® and Peg-Intron®

The term “ribavirin” (RBV) refers to the compound, 1-((2R,3R,4S,5R)-3,4-Dihydroxy-5-hydroxymethyl-tetrahydro-furan-2-yl)-1H-[1,2,4]triazole-3-carboxylic acid amide which is a synthetic, non-interferon-inducing, broad spectrum antiviral nucleoside analog and available under the names, Virazole® and Copegus®.

The terms “mericitabine”, “RG7128” and “RO5024048” are used interchangeably and refer to the diisobutyl ester prodrug of the cytosine nucleoside analog b-D-2′-Deoxy-2′-fluoro-2′-C-methycytidine which is an inhibitor of the HCV NS5B RNA polymerase.

Other HCV NS5B RNA polymerase inhibitors include “MK0608” (2′-C-Me-7-deaza-adenosine) from Isis Pharmaceuticals and Merck, “PSI-7977” ((S)-2-{[(1R,4R,5R)-5-(2,4-Dioxo-3,4-dihydro-2H-pyrimidin-1-yl)-4-(R)-fluoro-3-hydroxy-4-methyltetrahydrofuran-2-ylmethoxy]phenoxyphosphorylamino}propionic acid (S)-isopropyl ester) from Pharmasset, “INX 08189” (2-amino-6-methoxy-9-(2-C-methyl-b-D-ribofuranosyl) purine 50-O-[a-naphthyl-(2,2-dimethylpropoxy-L-alaninyl)]phosphate) from Inhibitex, “VX-222” (5-(3,3-Dimethyl-1-butyn-1-yl)-3-[(cis-4-hydroxycydohexyl)[(trans-4-methylcyclohexyl)carbonyl]amino]-2-thiophenecarboxylic acid,) from Vertex, “GS-9190” or “tegobuvir” (5H-Imidazo[4,5-c]pyridine, 5-[[6-[2,4-bis(trifluoromethyl)phenyl]-3-pyridazinyl]methyl]-2-(2-fluorophenyl)-5-({6-[2,4-bis(trifluoromethyl)phenyl]pyridazin-3-yl}methyl)-2-(2-fluorophenyl)-5H-imidazo[4,5-c]pyridine) from Gilead, “GSK625433” (D-Proline,1-[4-(1,1-dimethylethyl)-3-methoxybenzoyl]-4-(methoxymethyl)-2-(1H-pyrazol-1-ylmethyl)-5-(2-thiazolyl)-,(4R,5S)-rel-) from Glaxo SmithKline.

The term “triple therapy treatment” refers to a treatment regimen for HCV infected patients that comprises peginterferon, ribavirin and one or more direct acting antiviral agent. “Direct acting antiviral agents” exert specific antiviral effects independent of immune function. Examples of direct acting antiviral agents for HCV include but are not limited to NS3/4A protease inhibitors, NS5B polymerase inhibitors, NS5A inhibitors, IRES inhibitors and helicase inhibitors. One example of a triple therapy treatment comprises peginterferon, ribavirin, and a HCV NS5B polymerase inhibitor. Another example of a triple therapy treatment comprises peginterferon, ribavirin, and a HCV NS3/4A protease inhibitor. A further example of a triple therapy treatment comprises peginterferon, ribavirin, a HCV NS5B polymerase inhibitor, and a HCV NS3/4A protease inhibitor (which may also be referred to as “quadruple therapy treatment”.

For patients with chronic hepatitis C (CHC) the current recommended first line treatment, referred as “standard of care” or “SOC”, is pegylated interferon alpha in combination with ribavirin for a “duration of time” of 48 weeks in patients carrying genotype 1 or 4 virus and for 24 weeks in patients carrying genotype 2 or 3 virus. Combined treatment with ribavirin was found to be more effective than interferon alpha monotherapy in patients who relapsed after one or more courses of interferon alpha therapy, as well as in previously untreated patients. However, ribavirin exhibits significant side effects including teratogenicity and carcinogenicity. Furthermore, ribavirin causes hemolytic anemia requiring dose reduction or discontinuation of ribavirin therapy in approximately 10 to 20% of patients, which may be related to the accumulation of ribavirin triphosphate in erythrocytes. Therefore, to reduce treatment cost and the incidence of adverse events, it is desirable to tailor the treatment to a shorter duration while not compromising efficacy.

As used herein, the terms “allele” and “allelic variant” refer to alternative forms of a gene including introns, exons, intron/exon junctions and 3′ and/or 5′ untranslated regions that are associated with a gene or portions thereof. Generally, alleles occupy the same locus or position on homologous chromosomes. When a subject has two identical alleles of a gene, the subject is said to be homozygous for the gene or allele. When a subject has two different alleles of a gene, the subject is said to be heterozygous for the gene. Alleles of a specific gene can differ from each other in a single nucleotide, or several nucleotides, and can include substitutions, deletions, and insertions of nucleotides.

As used herein, the term “polymorphism” refers to the coexistence of more than one form of a nucleic acid, including exons and introns, or portion (e.g., allelic variant) thereof. A portion of a gene of which there are at least two different forms, i.e., two different nucleotide sequences, is referred to as a polymorphic region of a gene. A polymorphic region can be a single nucleotide, i.e. “single nucleotide polymorphism” or “SNP”, the identity of which differs in different alleles. A polymorphic region can also be several nucleotides long.

Numerous methods for the detection of polymorphisms are known and may be used in conjunction with the present invention. Generally, these include the identification of one or more mutations in the underlying nucleic acid sequence either directly (e.g., in situ hybridization) or indirectly (identifying changes to a secondary molecule, e.g., protein sequence or protein binding).

One well-known method for detecting polymorphisms is allele specific PCR or allele specific hybridization using either primers (PCR) or probes (hybridization) overlapping the mutation or polymorphic site and having about 5, 10, 20, 25, or 30 nucleotides around the mutation or polymorphic region. For use in a kit, e.g., several primers or probes capable of hybridizing specifically or preferentially to allelic variants, such as single nucleotide polymorphisms, are provided for the user or even attached to a solid phase support, e.g., a bead or chip.

The single nucleotide polymorphisms, “rs28416813”, “rs12979860”, and “rs810314” refer to SNPs identified by accession number in the database of SNPs (dbSNP, www.ncbi.nlm.nih.gov/SNP/) and are located on human chromosome 19 within the IL28b gene and its promoter region.

### EXAMPLES

### Example 1

### NV20536

### Study Design

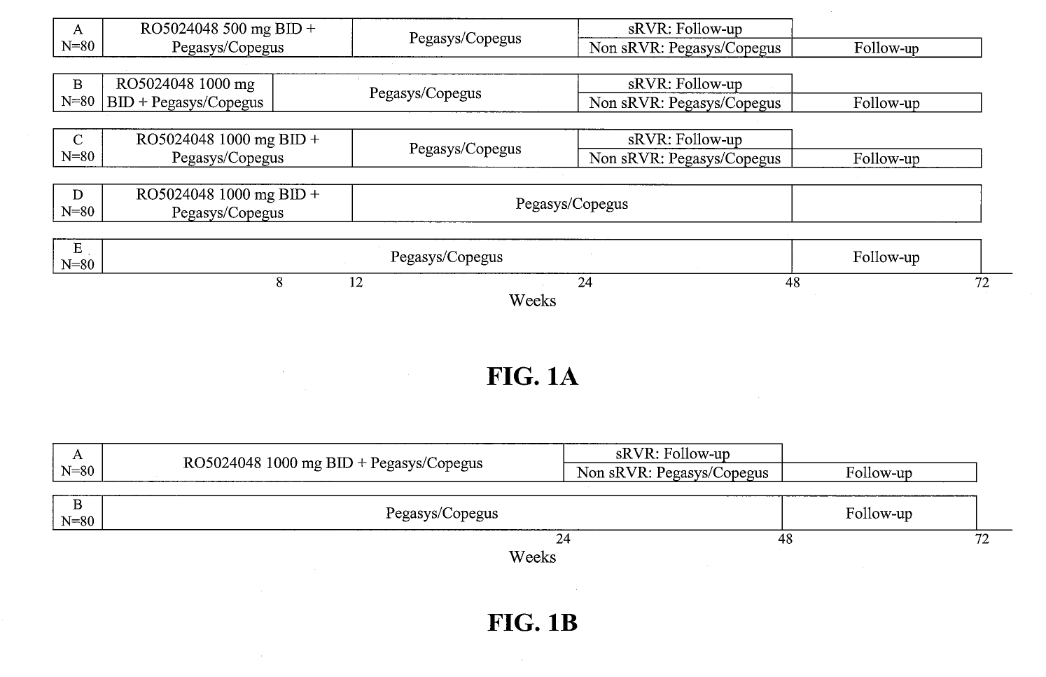

Study NV20536 was designed to determine the optimal dose (500 or 1000 mg twice daily [BID]) and duration (8 or 12 weeks) of RO5024048 in combination with PEG-IFN/RBV in treatment-naïve CHC genotype 1 or 4 patients. Patients enrolled in Study NV20536 were randomized to one of five treatment groups (FIG. 1A) (approximately 80 patients per treatment group). In the response-guided treatment groups, patients with a sustained RVR (sRVR, defined as undetectable HCV RNA from week 4-22) stopped all therapy at Week 24.

### Treatment Groups A and C:

Patients received double blinded experimental treatment with RO5024048 500 mg BID and standard of care treatment (SOC, Pegasys 180 μg sc qw+Copegus 1000 mg (<75 kg) or 1200 mg (≧75 kg) po qd) or RO5024048 1000 mg BID and SOC respectively, for 12 weeks followed by SOC for 12 weeks. Patients who achieve an RVR, defined as undetectable HCV RNA at week 4, and remain undetectable through week 22 stopped all treatment at week 24. Non-RVR patients received SOC for an additional 24 weeks for a total treatment duration of 48 weeks.

### Treatment Group B:

Patients received double blinded experimental treatment with RO5024048 1000 mg BID and SOC for 8 weeks followed by SOC for 16 weeks. Patients who achieve an RVR defined as undetectable HCV RNA at week 4, and remain undetectable through week 22 stopped all treatment at week 24. Non-RVR patients received SOC for an additional 24 weeks for a total treatment duration of 48 weeks.

### Treatment Group D:

Patients received double blinded experimental treatment with RO5024048 1000 mg BID and SOC for 12 weeks followed by SOC treatment for 36 weeks for a total treatment duration of 48 weeks.

### Treatment Group E:

Patients received double blinded placebo treatment and SOC for 12 weeks followed by SOC for 36 weeks for a total treatment duration of 48 weeks.
RCR Patient Population with IL28B Genotyping

The Roche Clinical Repository (RCR) is an optional program that requires separate consent; data are anonymized to assure patient privacy. The RCR DNA samples were used to determine IL28B status using TaqMan Real-Time PCR for rs12979860. Efficacy endpoints were summarized for the two subgroups, CC vs. non-CC (CT and TT).

### Results

A dose-dependent response was observed at Week 4 with 38% of patients achieving RVR (undetectable HCV RNA at week 4) in the 500-mg 12-week RVR-guided treatment group compared with 62% to 63% in the 1000-mg 8- and 12-week RVR-guided and the 1000-mg 12-week non-RVR-guided treatment groups. Eighteen percent of patients in the control group achieved RVR. There were higher complete early virological responses (cEVR), defined as undetectable HCV RNA at Week 12, of 68% to 87% in all experimental treatment groups compared with 49% in the control group. The Week 24 response rates in the experimental treatment groups (70% to 76%) were comparable to the response rate in the control group (73%). After week 24, the virologic response rate reflects a composite of different treatment influences in the response guided-arm: (1) the proportion of sRVR attained in that arm which determines the proportion who stop treatment at week 24, (2) the degree of relapse experienced by this sRVR subset associated with the short duration of treatment (24 weeks) and (3) the proportion of patients who have experienced viral rebound after stopping only RO524048 while remaining on treatment with PEG-IFN and RBV. In contrast, post week 24 virologic responses in both the non-RVR guided arm and the control arm represent on-treatment responses in all patients, and does not reflect the protocol-directed influence of treatment discontinuation at week 24 based on attainment of sRVR.

A total of 245 patients (˜60%) in Study NV20536 consented to RCR sampling and IL28B host genotyping at rs 12979860. Baseline characteristics as well as early virological response at Weeks 4 and 12 of the IL28B patient subset were considered representative of the larger population. Approximately 20% to 31% of the RCR-consented population demonstrate a CC genotype. The rates of cEVR in all treatment groups of the RCR-consented subset were comparable to the overall population.

An IL28B subset-analysis of the patients in each RVR-guided arm indicate that higher RO5024048 exposure (Arm C) is associated with a slightly greater increase in sRVR among patients with non-CC IL28B genotype. Non-CC patients comprised 50% (9/18) of patients achieving sRVR in Arm A compared to 60% (18/30) in Arm C Also, at 12 and 24 weeks followup after EOT, relapse in non-CC patients was higher in Arm C (13/18 and 14/18 respectively) than in Arm A (3/9 and 4/9 respectively) while in CC patients less pronounced increase in relapse was observed in arm C (4/12 and 4/12 respectively) compared to arm A (2/8 and 2/7 respectively)—a statistically significant interaction between treatment (arm A & arm C) and IL28B GT (CC & Non-CC) could not be confirmed due to low patient numbers) (Table1). Overall, in patients who achieve sRVR and stop all treatment after 24 weeks, CC patients demonstrate a higher SVR-12 and SVR-24 (67-90%) than do non-CC patients (22-67%).

TABLE 1

| | | Group A | | Group B | | Group C | |
|---|---|---|---|---|---|---|---|
| | | RO5024048 500 mg | | RO5024048 1000 mg | | RO5024048 1000 mg | |
| | | 12 wks | | 8 wks | | 12 wks | |
| | | P/R 24/48 wks | | P/R 24/48 wks | | P/R 24/48 wks | |
| | | RVR-guided | | RVR-guided | | RVR-guided | |
| | | N = 49 | | N = 49 | | N = 46 | |
| Total | sRVR, n (%) | 18 | (36.7%) | 23 | (46.9%) | 30 | (65.2%) |
| | SVR-12 | 12/17 | (70.6%)* | 14/23 | (60.9%) | 13/30 | (43.3%) |
| | n/N (%) | | | | | | |
| | SVR-24 | 11/16 | (68.8%)* | 11/22 | (50.0%)* | 12/30 | (40.0%) |
| | n/N (%) | | | | | | |
| CC | SVR-12 | 6/8 | (75.0%) | 9/10 | (90.0%) | 8/12 | (66.7%) |
| | n/N (%) | | | | | | |
| | SVR-24 | 6/7 | (85.7%) | 8/10 | (80.0%) | 8/12 | (66.7%) |
| | n/N (%) | | | | | | |
| NonCC | SVR-12 | 6/9 | (66.7%) | 5/13 | (38.5%) | 5/18 | (27.8%) |
| | n/N (%) | | | | | | |
| | SVR-24 | 5/9 | (55.6%) | 3/12 | (25.0%) | 4/18 | (22.2%) |
| | n/N (%) | | | | | | |

### Example 2

### NV22621

### Study Design

Study NV22621 was designed to determine the safety and efficacy of longer treatment duration (24 weeks) of 1000 mg BID RO5024048 in combination with PEG-IFN/RBV in treatment naïve CHC genotype 1 or 4 patients. Patients enrolled in Study NV22621 were randomized to one of two treatment groups (FIG. 1B) (approximately 80 patients per treatment group). In the response-guided treatment group, patients with a sustained RVR (sRVR, defined as undetectable HCV RNA from week 4-22) stopped all therapy at Week 24.

RCR Patient Population with IL28B Genotyping

The Roche Clinical Repository (RCR) is an optional program that requires separate consent; data are anonymized to assure patient privacy. The RCR DNA samples were used to determine IL28B status using TaqMan Real-Time PCR. Efficacy endpoints were summarized for the two subgroups, CC vs. non-CC (CT and TT).

### Results

A greater proportion of patients in the RVR-guided 1000-mg group achieved an RVR, cEVR, and week-24 virological response (63%, 89%, and 91%, respectively) compared with the control group (14%, 51%, and 62%, respectively). Approximately 70% of the patients in Study NV22621 consented to RCR sampling. Baseline characteristics as well as early virological response at weeks 4 and 12 for the IL28B-polymorphism rs 12979860 genotyped patient population were representative of the larger population. Thirty-five percent and 40% of the RCR-consented population were patients with a genotype of CC. The rates of cEVR in the treatment groups of the RCR-consented population were comparable to the overall ITT population (94% vs 89% and 47% vs 51%, for the 1000-mg and control groups, respectively). In the subset of patients who had sRVR in the RVR-guided 1000-mg arm, 80% of the patients with a genotype of CC and 72% of the patients with a non-CC genotype achieved SVR-12 (Table 2)

TABLE 2

| | | Group A | |
|---|---|---|---|
| | | RO5024048 1000 mg | |
| | | 24 wks | |
| | | P/R 24/48 wks RVR-guided | |
| | | N = 52 | |
| Total | sRVR, n (%) | 33 | (63.5%) |
| | SVR-12 | 24/32 | (75.0%)* |
| | n/N (%) | | |
| CC | SVR-12 | 12/15 | (80.0%) |
| | n/N (%) | | |
| NonCC | SVR-12 | 13/18 | (72.2%) |
| | n/N (%) | | |

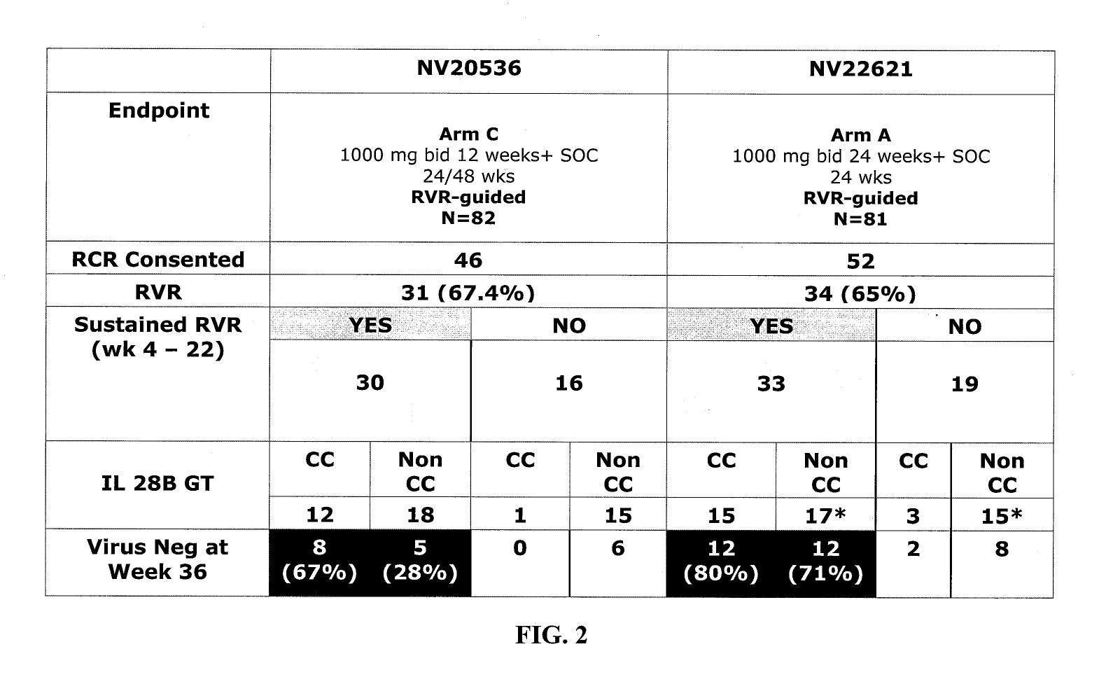

Comparison of Group C in NV20536 and Group A in NV22621 Group C patients in the NV20536 study who achieved sRVR received 1000 mg RO5024048 bid with SOC until Week 12 followed by SOC only until Week 24. In comparison, Group A patients in the NV22621 study who achieved sRVR received 1000 mg RO5024048 with SOC until Week 24. While patients with CC genotype at rs 12979860 that achieved SVR were not significantly different between these two groups (67% v 80%), patients with non-CC genotype who received RO5024048 for 24 weeks achieved SVR at a significantly higher proportion than the patients who received RO5024048 for only 12 weeks (28% v 71%, FIG. 2). Interestingly, the NV22621 study also showed that with 24 weeks of triple therapy treatment, patients with non-CC genotype reached a maximal rate (>90%) of undetectable viral load at weeks 20 to 24, and this rate decreased during the untreated follow-up period (data not shown). Therefore, it can be expected that the use of triple therapy for up to 48 weeks for patients with non-CC genotype can maximally maintain the benefit of undetectable viral load, i.e. SVR.

### Example 3

### Interim HCV Protease Inhibitor Study

In a protease inhibitor-containing triple regimen for treatment naïve, HCV genotype 1 patients, telaprevir/Advance trial T12PR, an eRVR-guided group demonstrated SVR of 75% (271/363). Subsequent analysis of patients with available IL28B genotype rs 12979860 information showed that SVR rates from CC, CT, and TT were 90% (45/50), 71% (48/68), and TT 73% (16/22) respectively (Jacobson I M et al, EASL 2011). In another protease inhibitor-containing triple regimen for the same indication, boceprevir/Sprint-2 trial response guided treatment (RGT) group demonstrated that the overall SVR was 63% (233/368). Further analysis of patients with available IL28B genotype information indicated that SVR rates from CC, CT, and TT were 82% (63/77), 65% 67/103), TT 55% (23/42) respectively (Poordad F et al, EASL 2011). Taken together, these 2 studies demonstrate that there is adequate room for further optimization of treatment in non-CC patients for maximal benefit. Therefore, a treatment regimen that consists of mericitabine/PegIFN/RBV and a protease inhibitor such as telaprevir and boceprevir can be chosen for patients with CT or TT genotypes who are presumably difficult-to-treat. Maximal incremental benefit of mericitabine may be achieved if given in this quadruple therapy treatment for up to a duration of 48 weeks.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

1. Lavanchy, D. Global Surveillance and Control of Hepatitis C: Report of a WHO Consultation. J Viral Hepatitis. 1999; 6:35-47.
2. Consensus Statement: EASL/International Consensus Conference on Hepatitis C. J. Hepatol. 1999; 31(Suppl 1):3-8.
3. Recommendations from the National Institutes of Health Consensus Development Conference Statement: Management of Hepatitis C: 2002. Hepatology November 2002; Vol 36 No 5:1039.
4. Fried M W, Shiffman M L, Reddy K R, Smith C, Marinos G, Goncales F L Jr, et al. 2002. Peginterferon alfa-2a plus rivavirin for chronic hepatitis C virus. NEJM Sep. 26, 2002; Vol 347 No13:975-982
5. Hadziyannis S J, Sette Jr. H, Morgan T R et al. Peginterferon alfa-2a and ribavirin combination therapy in chronic hepatitis C. Ann Int Med 2004 Vol 140:346-355.
6. Manns M P, McHutchison J G, Gordon S C, Rustgi V K, Shiffman M, Reindollar R, Goodman Z D, Koury K, Ling M, Albrecht J K. Peginterferon alfa-2b plus ribavirin compared with interferon alfa-2b plus ribavirin for initial treatment of chronic hepatitis C: a randomised trial. Lancet 2001; 358:958-965.
7. Report No. 1022744; 2006: Inhibition of RNA synthesis by by β-D-2′-deoxy-2′-fluoro-2′-C-methylcytidine (RO4995855) in the HCV Subgenomic Replicon System. Roche Palo Alto LCC.
8. RO5024048 Investigator's Brochure, November 2008.
9. PEG-IFN alfa-2a Investigator's Brochure, July, 2006.
10. PEGASYS Core Data Sheet Version 1.4, Jan. 19, 2005.
11. Sidwell R W, Robins R K, and Hillyard I W. Ribavirin: an antiviral agent. Pharmacol Ther 1979; 6:123-146.
12. Gilbert B E and Knight V. Minireview: Biochemistry and clinical applications for ribavirin. Antimicrob Agents Chemother. 1986; 30:201-205.
13. Dusheiko G, Main J, Thomas H et al. Ribavirin treatment for patients with chronic hepatitis C: Results of a placebo-controlled trial. J Hepatol 1996; 25:591-598.
14. Di Bisceglie A M, Conjeevaram H S, Fried M W et al. Ribavirin as therapy for chronic hepatitis C. A randomized, double-blind, placebo-controlled trial. Ann Int Med 1995; 123:897-903.
15. Conjeevaram H S, Fried M W, Jeffers L J, Terrault N A, Wiley-Lucas T E, Afdhal N, Brown R S, Belle S H, Hoofnagle J H, Kleiner D E, Howell C D. Peginterferon and ribavirin treatment in African American and Caucasian American patients with hepatitis C genotype 1. Gastroenterology 2006; 131:470-477.
16. Dienstag J L, McHutchison J G. American Gastroenterological Association technical review on the management of hepatitis C. Gastroenterology 2006; 130:231-264.
17. Missiha S, Heathcote J, Arenovich T, Khan K. Impact of asian race on response to combination therapy with peginterferon alfa-2a and ribavirin in chronic hepatitis C. Am J Gastroenterol 2007; 102:2181-2188.
18. Reddy K R, Messinger D, Popescu M, Hadziyannis S J. Peginterferon alpha-2a (40 kDa) and ribavirin: comparable rates of sustained virological response in sub-sets of older and younger HCV genotype 1 patients. J Viral Hepat 2009; 16:724-731.
19. Ferenci P, Fried M W, Shiffman M L, Smith C I, Marinos G, Goncales F L, Jr., Haussinger D, Diago M, Carosi G, Dhumeaux D, Craxi A, Chaneac M, Reddy K R. Predicting sustained virological responses in chronic hepatitis C patients treated with peginterferon alfa-2a (40 KD)/ribavirin. J Hepatol 2005; 43:425-433.
20. Martinot-Peignoux M, Maylin S, Moucari R, Ripault M P, Boyer N, Cardoso A C, Giuily N, Castelnau C, Pouteau M, Stern C, Auperin A, Bedossa P, Asselah T, Marcellin P. Virological response at 4 weeks to predict outcome of hepatitis C treatment with pegylated interferon and ribavirin. Antivir Ther 2009; 14:501-511.
21. Asselah T, Bieche I, Sabbagh A, Bedossa P, Moreau R, Valla D, Vidaud M, Marcellin P. Gene expression and hepatitis C virus infection. Gut 2009; 58:846-858.
22. Ge D, Fellay J, Thompson A J, Simon J S, Shianna K V, Urban T J, Heinzen E L, Qiu P, Bertelsen A H, Muir A J, Sulkowski M, McHutchison J G, Goldstein D B. Genetic variation in IL28B predicts hepatitis C treatment-induced viral clearance. Nature 2009; 461:399-401.
23. Suppiah V, Moldovan M, Ahlenstiel G, Berg T, Weltman M, Abate M L, Bassendine M, Spengler U, Dore G J, Powell E, Riordan S, Sheridan D, Smedile A, Fragomeli V, Muller T, Bahlo M, Stewart G J, Booth D R, George J. IL28B is associated with response to chronic hepatitis C interferon-alpha and ribavirin therapy. Nat Genet. 2009; 41:1100-1104.
24. Tanaka Y, Nishida N, Sugiyama M, Kurosaki M, Matsuura K, Sakamoto N, Nakagawa M, Korenaga M, Hino K, Hige S, Ito Y, Mita E, Tanaka E, Mochida S, Murawaki Y, Honda M, Sakai A, Hiasa Y, Nishiguchi S, Koike A, Sakaida I, Imamura M, Ito K, Yano K, Masaki N, Sugauchi F, Izumi N, Tokunaga K, Mizokami M. Genome-wide association of IL28B with response to pegylated interferon-alpha and ribavirin therapy for chronic hepatitis C. Nat Genet. 2009; 41:1105-1109.

1. A method of selecting a duration of time of triple therapy treatment for achieving sustained virological response in a human subject infected with HCV Genotype-1 or HCV Genotype-4,
wherein said triple therapy treatment comprises peginterferon, ribavirin, and a HCV NS5B polymerase inhibitor,
said method comprising:
providing a sample from said human subject and identifying the nucleotide present at single nucleotide polymorphism rs12979860,
wherein if said subject carries two C alleles at rs12979860, a duration of between 8 weeks and 12 weeks of said triple therapy treatment is selected, and
wherein if said subject does not carry two C alleles at rs12979860, a duration of at least 12 weeks and up to 48 weeks of said triple therapy treatment is selected.

2. The method of claim 1 wherein said triple therapy treatment is followed by standard of care (SOC) treatment wherein said SOC treatment comprises peginterferon and ribavirin.

3. The method of claim 1 wherein said HCV NS5B polymerase inhibitor is selected from MK0608, PSI-7977, INX 08189, VX-222, GS-9190, GSK625433, or mericitabine.

4. The method of claim 3 wherein said HCV NS5B polymerase inhibitor is mericitabine.

5. The method of claim 4 wherein mericitabine is provided at doses between 1000 mg and 2000 mg daily.

6. A method of selecting a duration of time of triple therapy treatment with or without subsequent standard of care (SOC) treatment for achieving sustained virological response in a human subject infected with HCV Genotype-1 or HCV Genotype-4,
wherein said triple therapy treatment comprises peginterferon, ribavirin, and a HCV NS5B polymerase inhibitor, and wherein said SOC treatment comprises peginterferon alfa-2a and ribavirin, said method comprising:
providing a sample from said human subject and identifying the nucleotide present at single nucleotide polymorphism rs12979860,
wherein if said subject carries two C alleles at rs12979860, a duration of between 8 weeks and 12 weeks of triple therapy treatment with or without subsequent 12 weeks of SOC treatment is selected, and
wherein if said subject does not carry two C alleles at rs12979860, a duration of at least 12 weeks and up to 48 weeks of triple therapy treatment with or without subsequent SOC treatment is selected.

7. The method of claim 6 further comprising:
monitoring whether said subject exhibits extended rapid virological response (eRVR) during triple therapy treatment or triple therapy treatment with subsequent SOC treatment.

8. The method of claim 7 wherein if said subject exhibits eRVR, and carries two C alleles at rs12979860, a duration of 12 weeks of triple therapy treatment with a subsequent 12 weeks of SOC treatment is selected, and wherein if said subject exhibits eRVR and does not carry two C alleles at rs12979860, a duration of at least 24 weeks and up to 48 weeks of triple therapy treatment with or without subsequent SOC treatment is selected.

9. The method of claim 6 wherein said HCV NS5B polymerase inhibitor is selected from MK0608, PSI-7977, INX 08189, VX-222, GS-9190, GSK625433, or mericitabine.

10. The method of claim 9 wherein said HCV NS5B polymerase inhibitor is mericitabine.

11. The method of claim 10 wherein mericitabine is provided at doses between 1000 mg and 2000 mg daily.

12. The method of claim 1 wherein if said subject does not carry two C alleles at rs12979860, a duration of between 12 weeks and 24 weeks of said triple therapy treatment is selected.

13. The method of claim 1 wherein said triple therapy treatment comprises peginterferon, ribavirin, a HCV NS5B polymerase inhibitor, and a HCV NS3/4A protease inhibitor.

14. The method of claim 6 wherein if said subject does not carry two C alleles at rs12979860, a duration of between 12 weeks and 24 weeks of triple therapy treatment with between 12 weeks and 24 weeks of subsequent SOC treatment is selected.

15. The method of claim 6 wherein said triple therapy treatment comprises peginterferon, ribavirin, a HCV NS5B polymerase inhibitor, and a HCV NS3/4A protease inhibitor
